(19) United States
(12) Patent Application Publication
(10) Pub. No.: US 2023/0172475 A1
(43) Pub. Date: Jun. 8, 2023

(54) SYSTEM AND METHOD FOR THE DETECTION OF DENTAL ANOMALIES USING MILLIMETER WAVE ANTENNA

(71) Applicants: Ian Petro, Waterford, PA (US); Abas Sabouni, Edwardsville, PA (US); Mahsa Khamechi, Edwardsville, PA (US)

(72) Inventors: Ian Petro, Waterford, PA (US); Abas Sabouni, Edwardsville, PA (US); Mahsa Khamechi, Edwardsville, PA (US)

(21) Appl. No.: 18/071,734

(22) Filed: Nov. 30, 2022

Related U.S. Application Data
(63) Continuation-in-part of application No. 63/286,121, filed on Dec. 6, 2021.

(51) Int. Cl. A61B 5/0507; H01Q 9/16; H01Q 21/06; A61B 5/00

(52) U.S. Cl. CPC A61B 5/0507; H01Q 9/16; H01Q 21/06; A61B 5/4547; A61B 5/682

(57) ABSTRACT

A system for the detection of dental anomalies including a casing, wherein the casing includes a plurality of antenna sources located along one side of the casing, and a plurality of sensors located along a second side of the casing, a mouthpiece substantially located between the first and second sides of the casing, a power module operatively connected to each of the plurality of antenna sources for providing electrical power to each of the antenna sources, and an analytics module operatively connected to each of the plurality of sensors for receiving and analyzing dental anomalies.

### CROSS-REFERENCE TO RELATED APPLICATION

This application is a continuation-in-part of U.S. Patent Application 63/286,121, filed on Dec. 6, 2021, the disclosure of which is hereby incorporated by reference in its entirety to provide continuity of disclosure to the extent such a disclosure is not inconsistent with the disclosure herein.

### FIELD OF THE INVENTION

The present invention is generally related to a system and method for the detection of dental anomalies (such as broken teeth, caries or the like) using a millimeter wave antenna.

### BACKGROUND OF THE INVENTION

Dental caries are permanently damaged areas in teeth (often referred to as cavities or tooth decay), roughly affecting 92% of adults aged 20 to 64 in the United States and about 2.3 billion adults worldwide. With limited amounts of affordable treatment options, the view shifts to prevention and early analysis.

Without insurance, dental x-rays cannot be performed frequently enough for low-income families due to a lack of an ability to afford the treatment. There is also the fear of x-ray radiation that imposes a barrier, thereby separating healthy adults from frequent testing. Furthermore, it is known that the X-ray technology can be very expensive to own, operate, and maintain, and the X-ray technology can have limits to its detection precision. As for the physical examination, it is known that the physical examination is limited to external imperfections. Therefore, a new form of testing must be created to satisfy these concerns of being financially accessible, providing a thorough examination, and providing low levels of radiation.

It is a purpose of this invention to fulfill these and other needs in the detecting of dental anomalies (such as broken teeth, caries or the like) art in a manner more apparent to the skilled artisan once given the following disclosure.

The preferred system and method for detecting dental anomalies (such as broken teeth, caries or the like), according to various embodiments of the present invention, offers the following advantages: ease of use; the use of millimeter wave antennas; the elimination of exposure to X-rays; reduced cost; improved precision in anomalies detection; and the ability to detect dental anomalies. In fact, in many of the preferred embodiments, these advantages are optimized to an extent that is considerably higher than heretofore achieved in prior, known systems and methods for detecting dental anomalies.

### BRIEF DESCRIPTION OF THE DRAWINGS

The above-mentioned features and steps of the invention and the manner of attaining them will become apparent, and the invention itself will be best understood by reference to the following description of the embodiments of the invention in conjunction with the accompanying drawings, wherein like characters represent like parts throughout the several views and in which:

FIGS. 1A and 1B are schematic illustrations of an imaging antenna being located on an outer side of each tooth, according to one embodiment of the present invention;

FIGS. 2A and 2B are schematic illustrations of an imaging antenna being located on an inner side of each tooth, according to one embodiment of the present invention;

FIGS. 3A and 3B are schematic illustrations of a single dipole antenna being located on an inner area of the patient's mouth, according to one embodiment of the present invention;

FIGS. 4A and 4B are schematic illustrations of multiple dipole antennas being located along an inner peripheral area of the patient's mouth, according to one embodiment of the present invention;

FIGS. 5A and 5B are schematic illustrations of multiple dipole antennas being located along an inner peripheral area of the patient's mouth and a reflective shield being located adjacent to the multiple dipole antennas, according to one embodiment of the present invention;

FIG. 6 is a top view of a system for the detection of dental anomalies using a millimeter wave antenna, according to one embodiment of the present invention;

FIG. 7 is an isometric view of the system for the detection of dental anomalies using a millimeter wave antenna, according to one embodiment of the present invention;

FIG. 8 is another top view of the system for the detection of dental anomalies using a millimeter wave antenna showing the locations of the antennas and the sensors, according to one embodiment of the present invention;

FIG. 9 is an exploded view of the system for the detection of dental anomalies using a millimeter wave antenna, according to one embodiment of the present invention;

FIG. 10 is a graphical illustration of a reference figure for the results from different shaped cavities for use FIGS. 11-15, according to the present invention;

FIG. 11 is a graphical illustration of the results from a centered sphere cavity, according to the present invention;

FIG. 12 is a graphical illustration of the results from a centered cylinder cavity, according to the present invention;

FIG. 13 is a graphical illustration of the results from no cavity on either end of the system, according to the present invention;

FIG. 14 is another graphical illustration of the results from no cavity on either end of the system, according to the present invention;

FIG. 15 is a graphical illustration of the results from a large, offset cube cavity, according to the present invention;

FIG. 16 is a graphical illustration of a reference figure for the results from FIGS. 17-44, according to the present invention;

FIG. 17 is a graphical illustration of the results for matching vs. cavity radius when the sphere cavity's origin is in the center of the tooth, according to the present invention;

FIG. 18 is a graphical illustration of the results for frequency response vs. cavity when the sphere cavity's origin is in the center of the tooth (0, 0, 0), according to the present invention;

FIG. 19 is a graphical illustration of the results for the absolute value of the matching difference between FIG. 17 and the response of no cavity present, according to the present invention;

FIG. 20 is a graphical illustration of the results for the absolute value of the frequency response difference between FIG. 18 and the response of no cavity present, according to the present invention;

FIG. 21 is a graphical illustration of the results for the matching vs. cavity radius when the sphere cavity's origin is shifted to −2.5 in the x direction (−2.5, 0, 0) with reference to FIG. 16, according to the present invention;

FIG. 22 is a graphical illustration of the results for frequency vs. cavity radius when the sphere cavity's origin is shifted to −2.5 in the x direction (−2.5, 0, 0) with reference to FIG. 16, according to the present invention;

FIG. 23 a graphical illustration of the results for the absolute value of the matching difference between FIG. 21 and the response of no cavity present, according to the present invention;

FIG. 24 a graphical illustration of the results for the absolute value of the frequency response difference between FIG. 22 and the response of no cavity present, according to the present invention;

FIG. 25 is a graphical illustration of the results for the matching vs. cavity radius when the sphere cavity's origin is shifted to +2.5 in the x direction (2.5, 0, 0) with reference to FIG. 16, according to the present invention;

FIG. 26 is a graphical illustration of the results for the frequency vs. cavity radius when the sphere cavity's origin is shifted to +2.5 in the x direction (2.5, 0, 0) with reference to FIG. 16, according to the present invention;

FIG. 27 is a graphical illustration of the results for the absolute value of the matching difference between FIG. 25 and the response of no cavity present, according to the present invention;

FIG. 28 is a graphical illustration of the results for the absolute value of the frequency response difference between FIG. 26 and the response of no cavity present, according to the present invention.

FIG. 29 is a graphical illustration of the results for the matching vs. cavity radius when the sphere cavity's origin is shifted to −2.5 in the y direction (0, −2.5, 0) with reference to FIG. 16, according to the present invention.

FIG. 30 is a graphical illustration of the results for the frequency vs. cavity radius when the sphere cavity's origin is shifted to −2.5 in the y direction (0, −2.5, 0) with reference to FIG. 16, according to the present invention.

FIG. 31 is a graphical illustration of the results for the absolute value of the matching difference between FIG. 29 and the response of no cavity present, according to the present invention.

FIG. 32 is a graphical illustration of the results for the absolute value of the frequency response difference between FIG. 30 and the response of no cavity present, according to the present invention.

FIG. 33 is a graphical illustration of the results for the matching vs. cavity radius when the sphere cavity's origin is shifted to +2.5 in the y direction (0, 2.5, 0) with reference to FIG. 16, according to the present invention.

FIG. 34 is a graphical illustration of the results for the frequency vs. cavity radius when the sphere cavity's origin is shifted to +2.5 in the y direction (0, 2.5, 0) with reference to FIG. 16, according to the present invention.

FIG. 35 is a graphical illustration of the results for the absolute value of the matching difference between FIG. 33 and the response of no cavity present, according to the present invention.

FIG. 36 is a graphical illustration of the results for the absolute value of the frequency response difference between FIG. 34 and the response of no cavity present, according to the present invention.

FIG. 37 is a graphical illustration of the results for the matching vs. cavity radius when the sphere cavity's origin is shifted to −2.5 in the z direction (0, 0, −2.5) with reference to FIG. 16, according to the present invention.

FIG. 38 is a graphical illustration of the results for the frequency vs. cavity radius when the sphere cavity's origin is shifted to −2.5 in the z direction (0, 0, −2.5) with reference to FIG. 16, according to the present invention.

FIG. 39 is a graphical illustration of the results for the absolute value of the matching difference between FIG. 37 and the response of no cavity present, according to the present invention.

FIG. 40 is a graphical illustration of the results for the absolute value of the frequency response difference between FIG. 38 and the response of no cavity present, according to the present invention.

FIG. 41 is a graphical illustration of the results for the matching vs. cavity radius when the sphere cavity's origin is shifted to +2.5 in the z direction (0, 0, 2.5) with reference to FIG. 16, according to the present invention.

FIG. 42 is a graphical illustration of the results for the frequency vs. cavity radius when the sphere cavity's origin is shifted to +2.5 in the z direction (0, 0, 2.5) with reference to FIG. 16, according to the present invention.

FIG. 43 is a graphical illustration of the results for the absolute value of the matching difference between FIG. 41 and the response of no cavity present, according to the present invention.

FIG. 44 is a graphical illustration of the results for the absolute value of the frequency response difference between FIG. 42 and the response of no cavity present, according to the present invention.

FIG. 45 is a graphical illustration of results for the absolute matching difference between a small cavity and no cavity versus frequency whenever the cavity is centered in tooth according to the present invention.

### DETAILED DESCRIPTION OF THE PREFERRED EMBODIMENTS OF THE INVENTION

In the past two decades, microwave imaging has received intense attention for biomedical applications. Microwave imaging offers many attractive features, such as having a low health risk, being noninvasive, simple to perform, cost-effective, and causing minimal discomfort.

In microwave imaging, to reconstruct images with high resolution, high frequency is desired. On the other hand, depending on the application, the penetration depth may decrease as the frequency increases. Therefore, the higher microwave frequency range (millimeter range) can only be used for those imaging applications such as security imaging, automotive radar imaging, ship, aircraft, and spacecraft imaging which the image of the surface of the object is required. The millimeter wave (mmW) imaging of the present invention has the advantage of super-resolution imaging with minimal. Furthermore, the present invention discloses a portable imaging device that uses mmW frequency to detect dental anomalies (such as broken teeth, caries or the like) at the very early stages of development.

In order to address the shortcomings of the prior, known systems and methods for detecting dental anomalies, it would be desirable to utilize a system and method for the detection of dental anomalies that employs a millimeter wave antenna. In particular, it would be desirable to be able to identify anomalies that exist in individual teeth. Also, it would be desirable to be able to determine the level of severity of the detected dental anomalies.

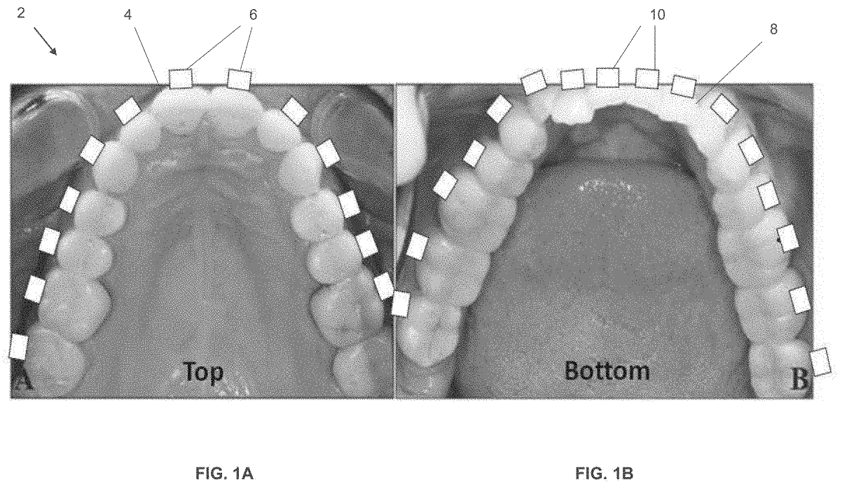

To be able to identify anomalies that exist in individual teeth and determine the level of severity of the detected dental anomalies, reference is made now to FIGS. 1A and 1B, where there are illustrated an imaging antenna being located on an outer side of each tooth, according to one embodiment of the present invention.

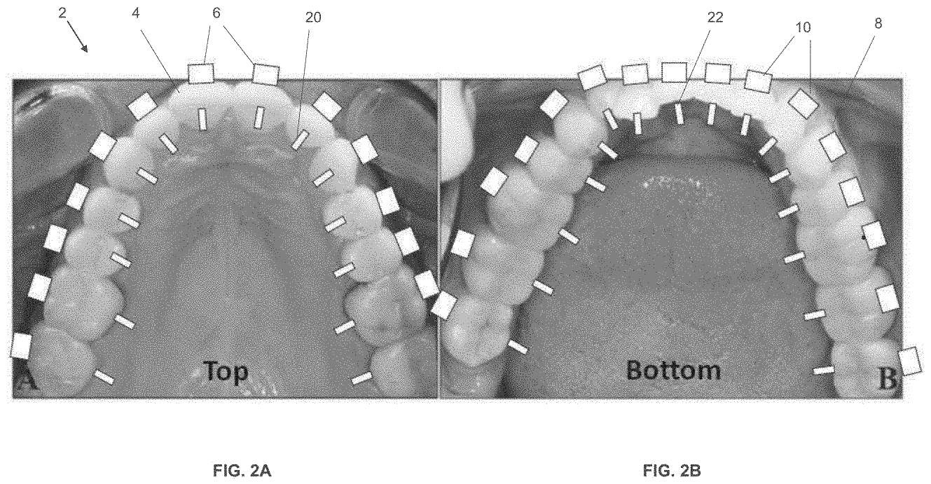

With respect to FIGS. 1A and 1B, FIGS. 1A and 2B are schematic illustrations of an imaging antenna being located on an outer side of each tooth, according to one embodiment of the present invention. In particular, FIGS. 1A and 1B show the basic structure of imaging system 2 for the detection of dental anomalies using a millimeter wave antenna. As shown in FIG. 1A and 1B, system 2 includes, in part, top teeth 4, a plurality of top antennas 6, bottom teeth 8, and a plurality of bottom antennas 10.

Because there are two rows of teeth (top teeth 4 and bottom teeth 8), there must be two rows of imaging antenna 6 and 10. In one embodiment, top antennas 6 will only be usable with the top teeth 4, and the bottom antennas 10 will only be usable with the bottom teeth 8. In one embodiment, the antennas 6 and 10, preferably will have dimentions of 4×6×0.5 millimeter or smaller. Also, antennas 6 and 10 should be constructed so as to be able to detect microwave (μwave) to millimeter wave (mmW) frequencies in order to detect dental anomalies. It is to be understood that microwave (μwave) is the band of spectrum with wavelengths between 100 millimeters (3 GHz frequency) and 10 millimeters (30 GHz frequency, and the millimeter wave (mmW), also known as millimeter band, is the band of spectrum with wavelengths between 10 millimeters (30 GHz frequency) and 1 millimeter (300 GHz frequency). It is also known as the extremely high frequency (EHF) band. This frequency operating band has been proven to be safe, as it is the primary frequency band used in current 5G cellular communications. Finally, in another embodiment, antennas 6 and 10 can be broadband millimeter wave (mmW) antennas that can detect frequencies between 3 GHz and 300 GHz.

A unique aspect of the present invention is that the relatively small size of antennas 6 and 8 is extremely advantageous due to the average dimentions of adult teeth being greater than 6 mm. Another unique aspect of the present invention is that while a possible issue can exist regarding the lower insizers, as they are the smallest teeth in the mouth, simulations of the present invention have shown no technical issues, as will be discussed in greater detail later.

With respect to FIGS. 2A and 2B, there is illustrated another embodiment of the system 2 for the detection of dental anomalies using a millimeter wave antenna. As shown in FIGS. 2A and 2B, the system 2 includes, in part, top teeth 4, top antennas 6, top transmitters 20, bottom teeth 8, bottom antennas 10, and bottom transmitters 22. In one embodiment, top transmitters 20 and bottom transmitters 22, preferably, are constructed in a similar manner to top antennas 6 and bottom antennas 10 except that the top transmitters 20 are turned 90° with respect to top antennas 6 on the back side of each top tooth 4 to supply a signal to top antennas 6 for analysis of top teeth 6 and bottom transmitters 22 are turned 90° with respect to bottom antennas 10 on the back side of each bottom tooth 8 to supply a signal to bottom antennas 10 for analysis of bottom teeth 8.

Another unique aspect of the present invention is the orientation of top antennas 8 with respect to top transmitters 20 and bottom antennas 8 with respect to bottom transmitters 22. If both the antenna and the transmitter are in the same orientation, the sensitivity of system 2 to abnormalities in the teeth 6 and 8 was very low. By turning the transmitters 20 and 22 by 90°, and having transmitters 20 and 22 activated at the same time, abnormalities in teeth 4 and 8 as small as 0.2 mm in diameter were able to be detected.

Another unique aspect of the present invention is that there are several other considerations regarding the system 2 that need to be taken into account. Firstly, the inside edge of human teeth are concaved, and this can make appropriate alignment between antennas 6 and transmitters 20 and antennas 10 and transmitters 20, respectively, difficult. Secondly, not all smiles are created equaly, so being able to make system 2 flexible enough to adhere to a variety of crooked teeth 4 and/or 8 could become problematic. Thirdly, if there are 28-32 measureable teeth 4, 8 in the adult human mouth, that would require 56-64 antenna/transmitter per unit. This would make manufacturing, powering, and measurement very costly.

In another embodiment of system 2, it may be desirable for the system 2 in which the transmitters 20 and 22 were eliminated, thereby reducing the number of required antenna and transmitter by half. Therefore, it may be desired to improve the system 2 through the use of a singular source dipole structure antenna that could provide a signal that covers the entire mouth rather than having an individual transmitter per tooth.

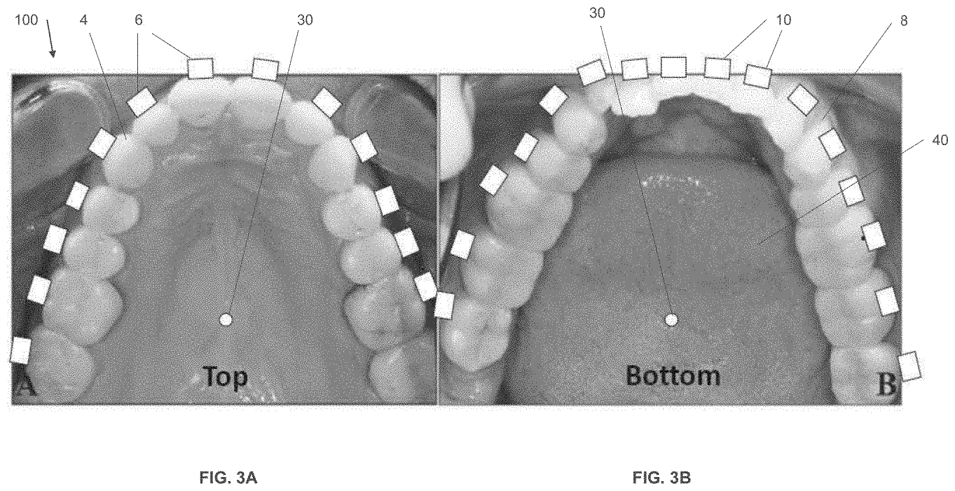

With respect to FIGS. 3A and 3B, there is illustrated a system 100 for the detection of dental anomalies using a millimeter wave antenna and a single dipole antenna that is located on an inner area of the patient's mouth. In particular, the system 100 includes, in part, upper teeth 4, upper antennas 6, bottom teeth 8, bottom antennas 10, and single dipole antenna 30.

Regarding single dipole antenna 30, in one embodiment, single dipole antenna 30 is a type of radio frequency (RF) antenna, consisting of two conductive elements such as rods or wires. The dipole is any one of the varieties of antenna that can produce a radiation pattern approximating that of an elementary electric dipole.

A dipole antenna 30 is a more desireable sourse for many reasons. Firstly, it would reduce the over all number of required antenna by almost half. Secondly, due to the orientation of dipole antenna 30, the bulk majority of the radiated energy is directed out (toward the teeth 4 and 8) with almost no radiation directly up (toward the brain) or down (into the jaw). Thirdly, the size of dipole 30 is inversly proportional to the frequency of the signal. This means that the higher the frequency, the smaller the physical size of the dipole antenna 30. In an application where higher frequency is necessary, a property like this is desireable. Furthermore, this system 100 should more easily comply with the Federal Communications Commission (FCC) regulations for specific absoption rate (SAR). SAR is a measure of the rate at which energy is absorbed per unit mass by a human body when exposed to a radio frequency (RF) electromagnetic field. All commercial and non-commercial products alike must be below specific SAR values (dependent upon their particular application, such as a ‘controlled’ and ‘not-controlled’ exposures).

It has been determined that the placement of the dipole antenna 30 is important. When looking at an open mouth in a mirror (or even at all of the presented Figures), it can be seen that the tongue 40 lies almost flush against the bottom teeth 8. For best results with a singular source dipole 30, it should be placed nearest to the center of the mouth as possible, as shown in FIGS. 3A and 3B.

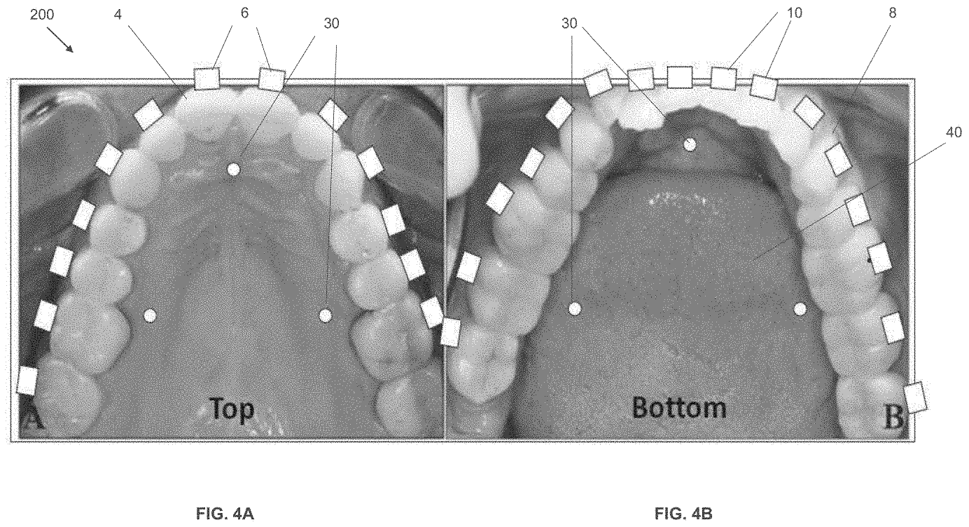

In order to address any possible issues with respect to the placement of dipole antenna 30, attention is directed to FIGS. 4A and 4B which ilustrates sytem 200 having a triangular array of dipole antennas 30. In this embodiment, the triangular array of dipole antennas 30 are arranged in a way that more closely matches the natural curvature of the teeth 4 and 8 in the mouth.

As shown in FIGS. 4A and 4B, system 200 includes, in part, three (3) dipole antennas 30 that, in one embodiment, are located equadistant across the mouth in order to contour to the shape of the jaw and teeth 4 and 8, and the placement of the tongue 40. It is to be understood that in system 200, due to the loading effect of the teeth 4 and 8 on the dipole antennas 30, the reflected energy would be sent to the back of the throat. This concentration of energy would result in difficult satisfaction of FCC SAR regulations.

Another unique aspect of the present invention is that to alleviate the possible reflection of energy back towards the throat, a reflective casing can be placed around the back side of the dipole antennas 30 to reflect most all of the energy towards the teeth 4 and 8, thereby resulting in even higher levels of accuracy in measurement readings and an improved ability to comply with FCC SAR regulations.

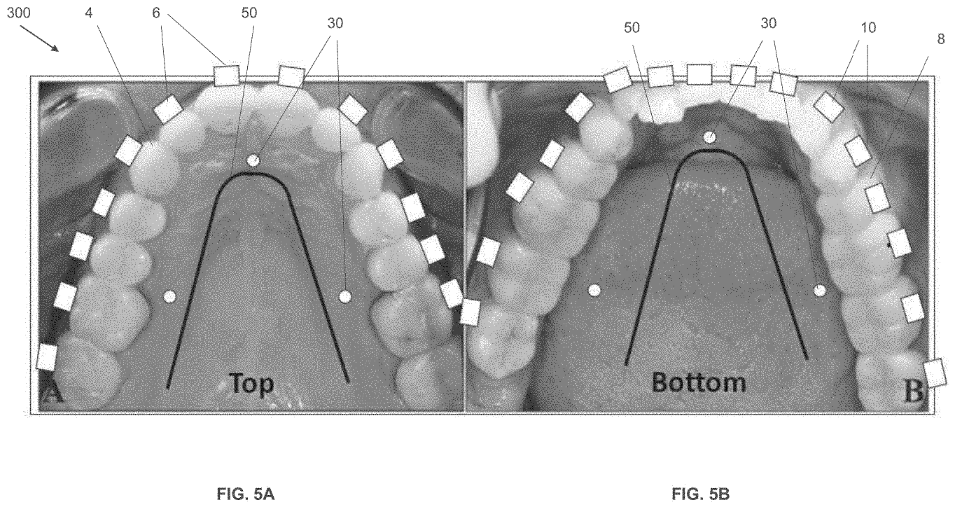

In order to address the undesirability of reflecting reflected energy towards the back of the throat, attention is directed towards FIGS. 5A and 5B. As shown in FIGS. 5A and 5B, system 300 for the detection of dental anomalies using a millimeter wave antenna by using multiple dipole antennas and a reflective casing 50 located on an area of the patient's mouth is illustrated. In one embodiment, the system 300, includes, in part, the triangular array of dipole antennas 30 and a reflective casing 50. Preferably, reflective casing 50 is constructed of a non-toxic, light weight, and highly reflective material when used in the respective frequency range.

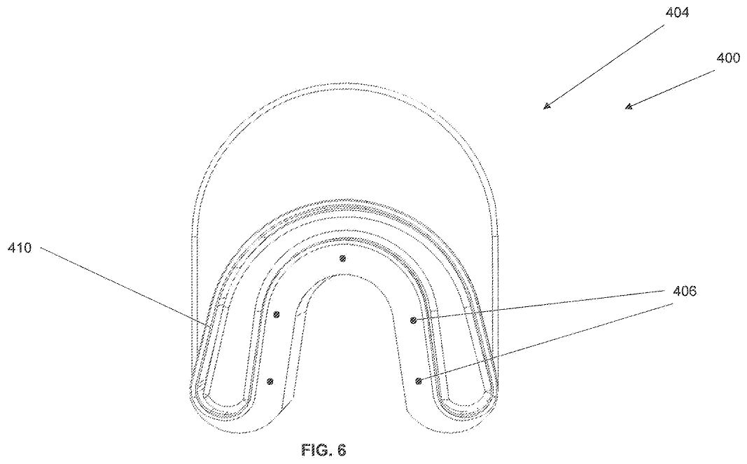

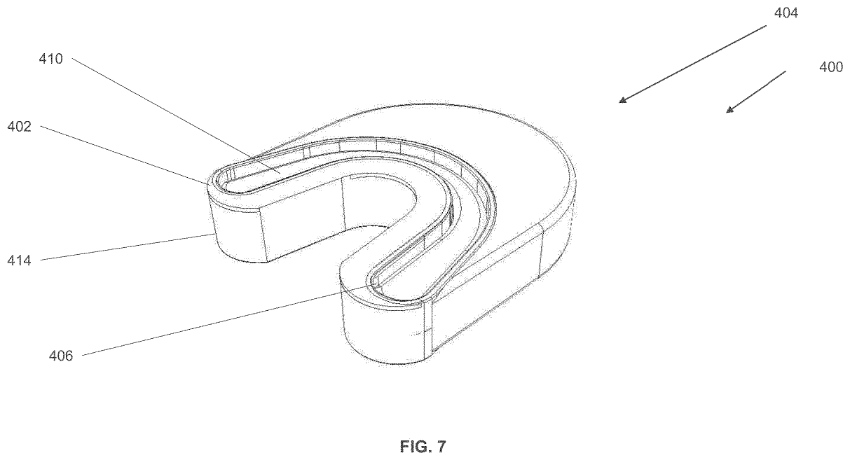

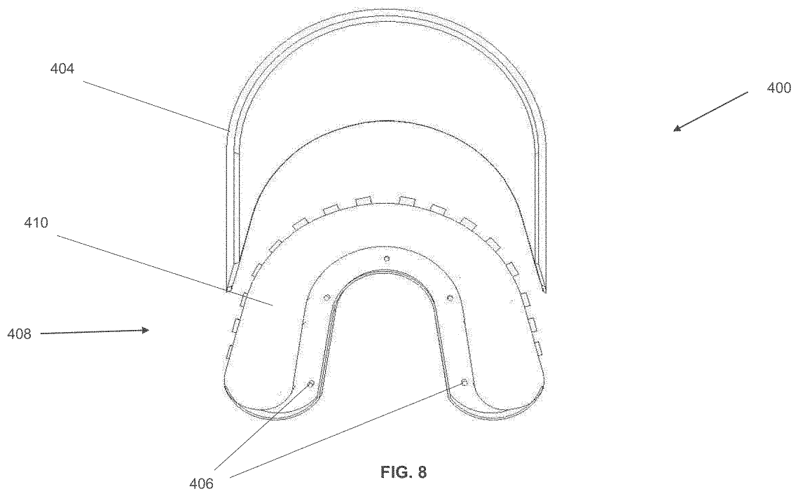

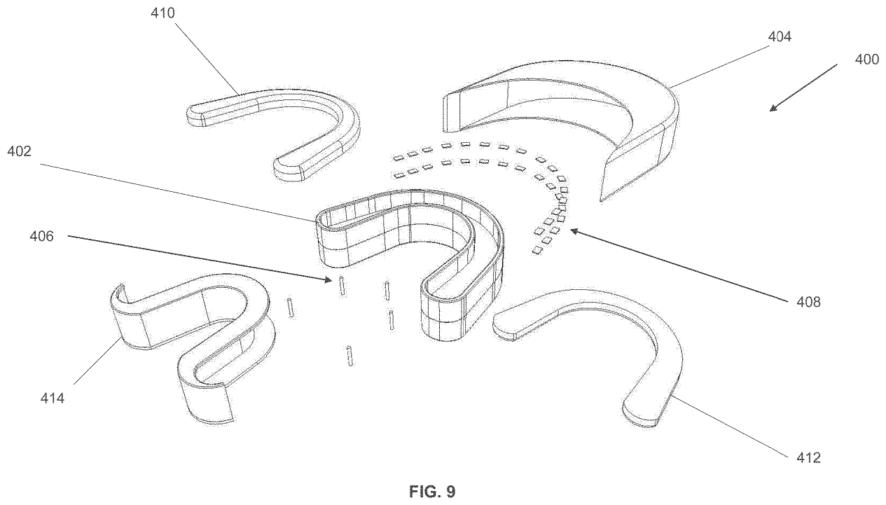

Given the above background and the fact that there are many parts associated with one system, the question of housing/storage of components arises. With this in mind, attention is directed to FIGS. 6-9. As shown in FIGS. 6-9, system 400 is illustrated. System 400 includes, in part, mouthpiece 402, electronics housing and reflective casing 404, dipole antennas sources 406, sensors 408, top teeth moldable mouthpiece 410, bottom teeth moldable mouthpiece 412, and rear reflective casing 414 (located in the center mouthpiece 402). In one embodiment, mouthpiece 402, top and bottom teeth moldable mouthpiece 410 and 412, and electronics housing 404 are constructed of any suitable UV-resistant, moldable, medical grade polymeric material similar to ethylene vinyl acetate (EVA) or thermopolymer which are most commonly found in present day sport and orthadontic mouthguards. Reflective casing 414 (and part of 404) are constructed of any appropriate reflective material that is suted for best operation at this particular frequency range.

Regarding dipole antennas sources 406, dipole antennas sources 406 preferably, are constructed in a similar manner as dipole antennas 30 (FIGS. 4A, 4B, 5A, and 5B). In one embodiment, dipole antenna sources 406 are located on reflective casing 414 (FIGS. 6-9) in a triagular array or pattern. Also, dipole antenna sources 406 are housed in a biomedically safe foam to keep their position constant to the mouthpiece 402. When working at high frequencies, foam is “invisible” to the antenna; therefore, the wave shape, amplitude, and reflections will not be affected. The number of transmitting dipoles and their locations in the devices are subject to change with further research and development. The arc pattern is utilized to better fit the curvature of the mouth.

Regarding sensors 408, sensors 408 preferably, are constructed in a similar manner as antennas 6 and 10 (FIGS. 5A and 5B). In one embodiment, sensors 408 are located around a periphery on an outer side of electronics housing 404 (FIGS. 6-9). Also, sensors 408 are partially implanted into the mouthpiece 402 so that the position relativly does not change. The center of each receiving antenna are be located nearest the center of the average tooth position in the human mouth. The receiving antenna are located nearest the electronics of the system to acieve the highest speed and accuracy of the data processing. In order to protect the encased electronics, another reflection shield will be incorperated in to the electronics housing 404.

In one embodiment, the mouthpiece 402 (including the internal components 404, 406, 408, 414) is to be provided with a customizeable mouthpiece assembly that include top moldable teeth mouthpiece 410, bottom moldable teeth mouthpiece 412. In this manner, the mouthpiece 402 can be made univeral to fit multiple different users by offering removable custom tooth molds to fit their exact teeth alignment. The unmolded pieces will have to be molded in a surrogate mouthpiece then transferred into the measurement mouthpiece 402. This allowes multiple users to use the same mouthpiece to help keep costs lower.

It is to be understood that conventional electronic wiring (not shown) can be attached to the dipole antenna sources 406 and sensors 408 by conventional techniques. Furthermore, the electonic wiring can also be attached to conventional power sources (not shown) in order to power the dipole antenna sources 406. Finally, the electronic wiring can be attached to conventional analytical equipment (not shown) in order to create and analyze the dental carries information received from the sensors 408. For initial research and development, high-frequency AC voltage power supply will feed the dipole antenna at a high frequency. The sensors output will be connected with a flex cable (not shown) to an N by one switch (not shown), and the output of the switch will be connected to a network analyzer using a flex cable. The switch will read the output of each sensor, and the data (scattering parameters) will be collected by the network analyzer (not shown). A computer (not shown) reads this data from the USB port (not shown) of the network analyzer and uses software programming to analyze the dental carrier information. When further research and development is done, the measurement electronics will be located in the electronics housing 404, which will send the results to a smartphone or similar device (not shown) for processing.

Another unique aspect of the present invention is the use of the mouthpiece 402 and the reflective casing 414 having the dipole antennas sources 406 and sensors 408. For example, the shape of moutpiece 402 is common with athletes and non-athletes alike. In particular, as is known in the mouthpiece art, a surrogate mouthpiece (like 402) typically is boiled and the user simply bites into the soft mouthpiece 410 and 412 in order to mold the pieces to the particular bite of the user to then be transferred in to the measurement mouthpiece 402.

Another unique aspect of the present invention is the location of the dipole antennas sources 406 with reflective casing 414 and sensors 408 adjacent to the processing electronics in the elctronics housing 404. As shown in FIGS. 6-9, placing the antenna sources 406 arbitrarily close to the position of the average tooth placement would make the system 400 more universally usable. The user could then mold the mouthpiece components 410 and 412 to match their particular bite, then insert it into the mouthpiece 402. In this embodiment, the system 400 is capable of being usable by more than one user, thereby making this sytem 400 attractive to the dental and/or private industry.

Furthermore, with this system 400, there is a reduction of components, ease of wiring, and explotiation of physical limitations. The system 400 is a common shape, and would appeal to many as well as be useable by more than one person.

In another embodiment, the system 400 can also include teeth whitining and wireless result capabilities. Also, by extending the system 400 out past the lips, there can be space included for all of the drive/measurement circuitry (not shown), and a battery (not shown), thereby making the system 400 atonomous and easily transportable. Furthermore, this space can be modified to include preexisting teeth whitening technology, as well as bluetooth capabilities to export data for compilation outside of the system 400, such as on a smartphone.

In still another embodiment, having a mobile application capable of processing results off system 400 will offer many advantages. First, it will make the make the overall size of system 400 smaller. The freed space can be used to either increase battery size or shink the entire form factor of system 400. Secondly, when the results are sent to the mobile application to be processed, the mobile application can reference an online database for the most up-to-date information to make the readings from sensors 408 as accurate as possible.

### Testing Results

In order to prove the efficacy of the present invention, the following test results are being provided.

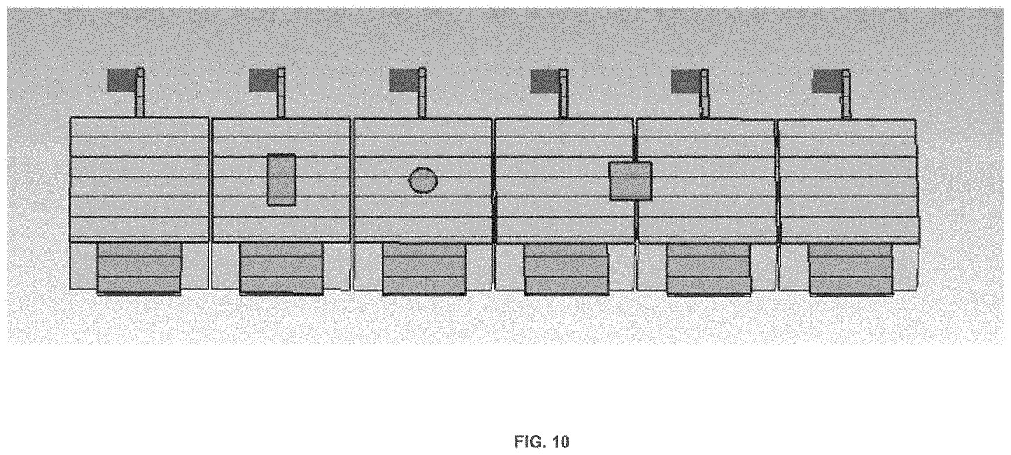

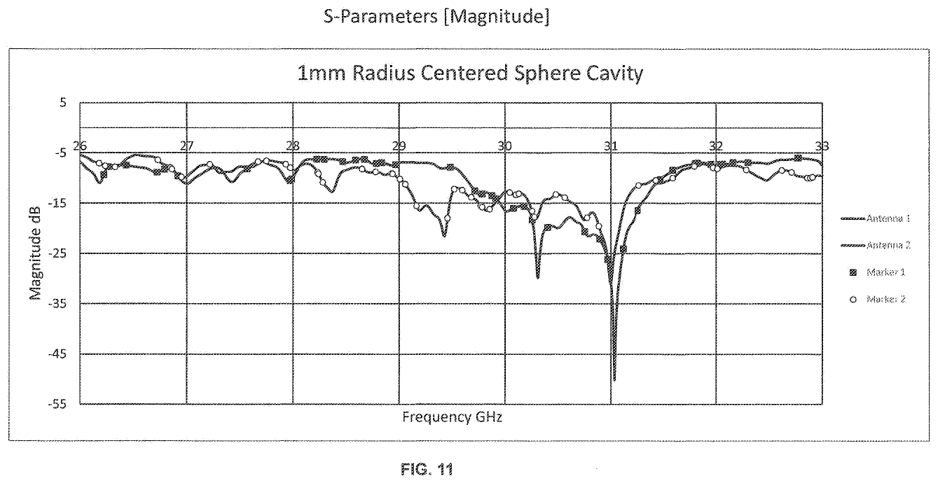

FIG. 10 is a graphical illustration of a reference figure for the results from different shaped cavities for FIGS. 11-15, according to the present invention. In particular, it is to be understood that individual tooth simulation is important, but when implemented in a real situation, the present system will need to be able to scan an entire mouth. Furthermore, it is to be understood that not all dental anomalies are perfect spheres. Finally, it is to be understood that adjacent antenna sources 406 will have slight interferences from their neighbors.

FIG. 11 is a graphical illustration of the results from a centered sphere cavity, according to the present invention. In particular, FIG. 11 shows various radial sphere cavities. Furthermore, the graphical relationship between the antenna one (simulation 1) and antenna two (simulation 2) scattering parameters with respect to FIG. 10 is shown. For example, antennas one (simulation 1) and two (simulation 2) show results that characteristically differ from the baseline, thereby indicating a cavity.

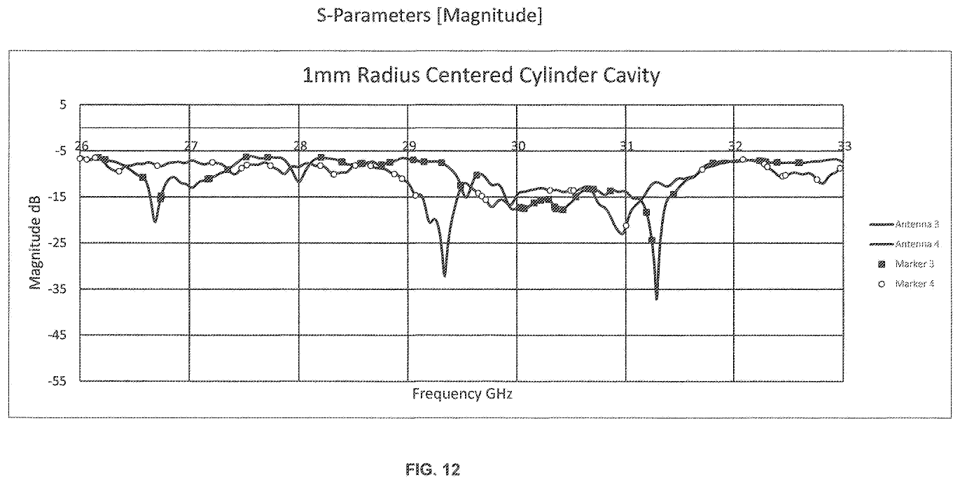

FIG. 12 is a graphical illustration of the results from a centered cylinder cavity, according to the present invention. In particular, the graphical relationship between the antenna three (simulation 3) and antenna four (simulation 4) scattering parameters with respect to FIG. 10 is shown. It is to be understood that antennas three (simulation 3) and four (simulation 4) show results that characteristically differ from the baseline, thereby indicating a cavity.

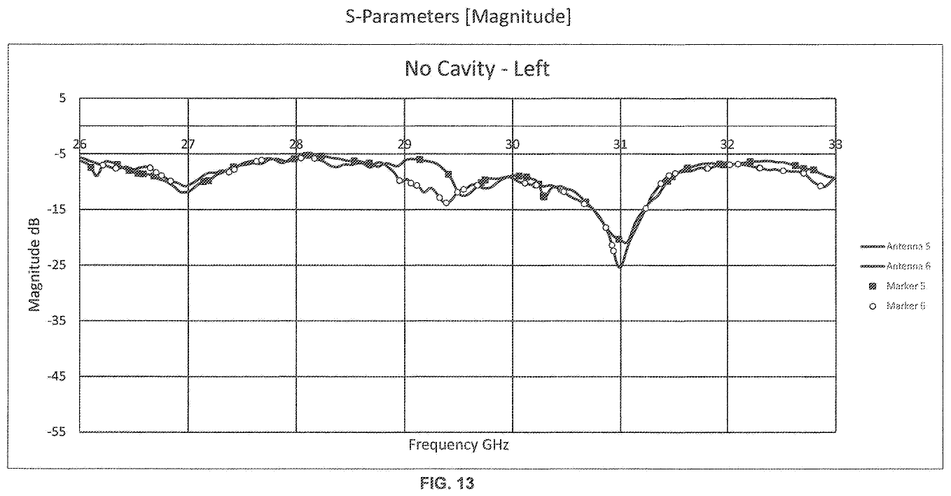

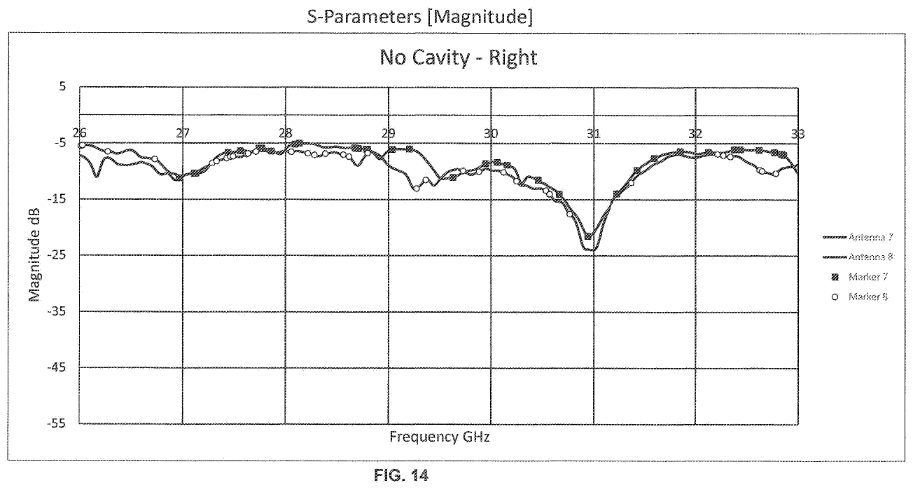

FIG. 13 is a graphical illustration of the results from no cavity on either end of the system, according to the present invention. In particular, FIG. 13 shows no cavities on the left side with respect to FIG. 10. In particular, the graphical relationship between the antenna five (simulation 5) and antenna six (simulation 6) scattering parameters with respect to FIG. 10 is shown. It is to be understood that FIG. 13 and FIG. 14 are similar.

FIG. 14 is another graphical illustration of the results from no cavity on either end of the system, according to the present invention. In particular, FIG. 14 shows no cavities on the right side with respect to FIG. 10. In particular, the graphical relationship between the antenna seven (simulation 7) and antenna eight (simulation 8) scattering parameters with respect to FIG. 10 is shown.

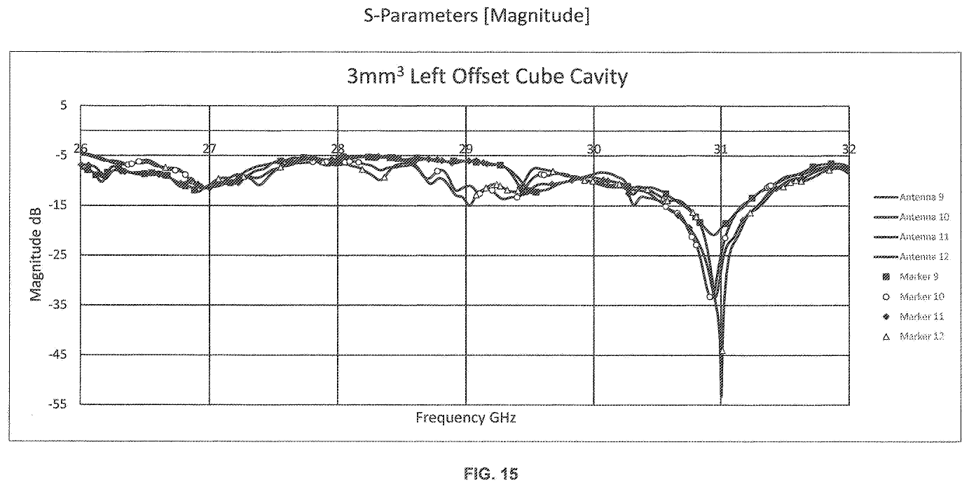

FIG. 15 is a graphical illustration of the results from a large, offset cube cavity, according to the present invention. In particular, FIG. 15 shows an off-center cube cavity. Furthermore, the graphical relationship between the antenna nine (simulation 9), antenna ten (simulation 10), antenna eleven (simulation 11), and antenna twelve (simulation 12) scattering parameters with respect to FIG. 10 is shown. It is to be understood that antenna nine (simulation 9) shares properties seen in antenna five (simulation 5) and antenna seven (simulation 7) with respect to FIG. 14 and FIG. 15, respectfully. It is to be understood that antennas ten (simulation 10), eleven (simulation 11), and twelve (simulation 12) show results that characteristically differ from the baseline, thereby indicating a cavity. It is important to note that the side that the cube was the heaviest on was the side that matched the most. The side the cube was less on looked almost like the results from FIGS. 13 and 14.

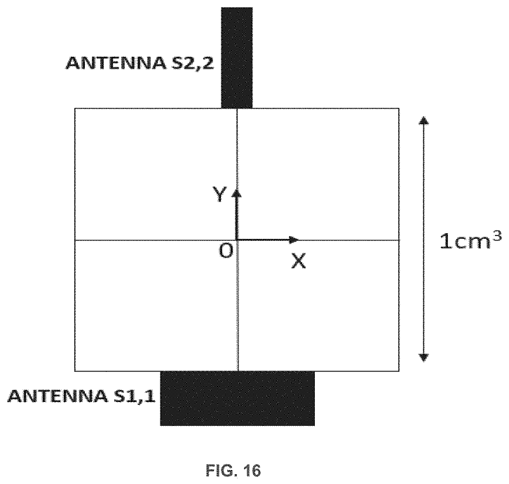

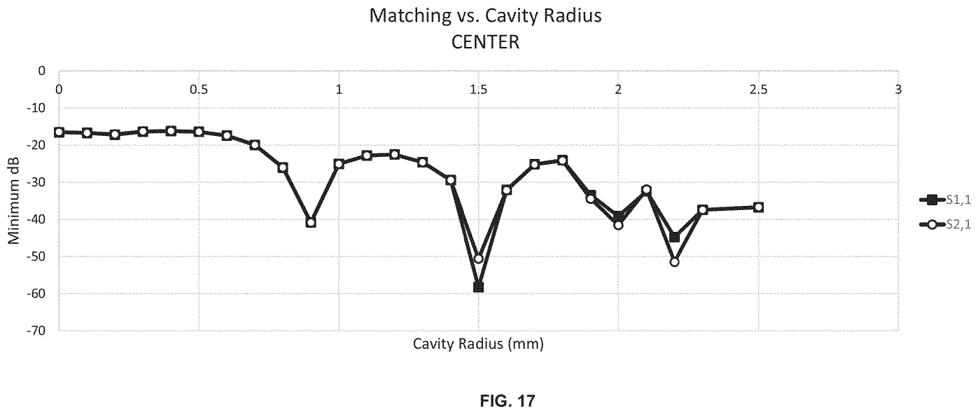

FIG. 16 is a graphical illustration of a reference figure for the results in FIGS. 17-44, according to the present invention. In particular, FIG. 16 is an application of an S-parameter and shows a graphical representation of the simulated origin referred to as antennas 1,1 and 2,2, as discussed below. In particular, scanning a model of a tooth with no cavities gives a baseline to compare against when a cavity is introduced. It is to be understood that the origin is in the center of the tooth. Whenever the cavity gets moved around the tooth, the X, Y, Z coordinates are used to show what direction the cavity moves. Results from antennae 1,1 and 2,2 come from the various antenna discussed in FIGS. 17-44.

FIG. 17 is a graphical illustration of the results for matching versus cavity radius when the sphere cavity's origin is in the center of the tooth, according to the present invention. It can be seen that the amount of matching between antennae 1,1 and 2,2 are almost exactly the same as the size of the cavity increases. This signifies that the cavity must be equidistant from both antennae if they are seeing the same amount of reflected energy.

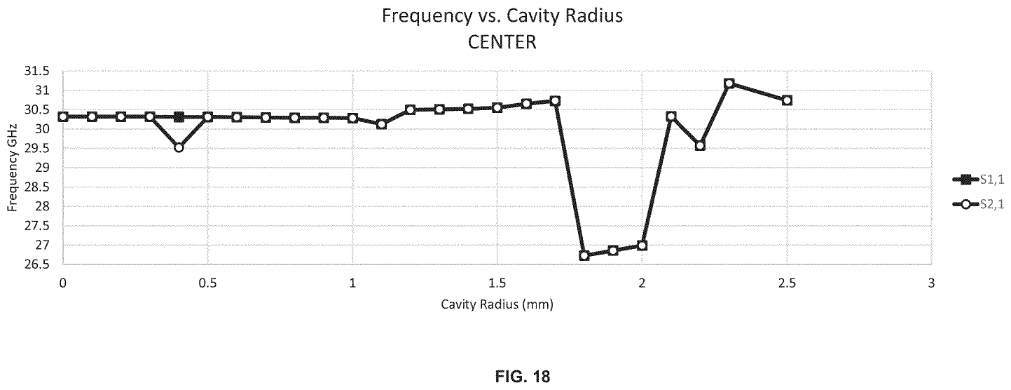

FIG. 18 is a graphical illustration of the results for frequency response versus cavity when the sphere cavity's origin is in the center of the tooth (0, 0, 0), according to the present invention. At this scale, this simulation shows that once the cavity reaches about 1.7 mm in diameter, the system becomes very sensitive to it; however, actual data analysis shows significant differences between the values at very small ranges. Further tuning of the system can make the sensitivity towards the center of the tooth much higher. This is important when identifying potential root issues in teeth.

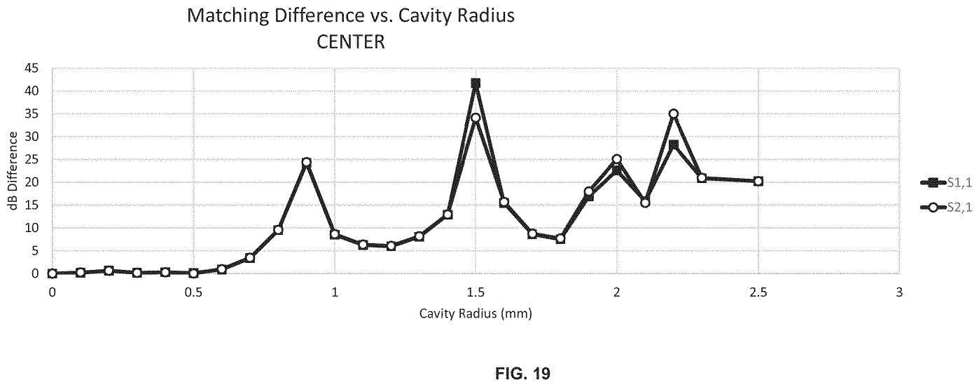

FIG. 19 is a graphical illustration of the results for the absolute value of the matching difference between FIG. 17 and the response of no cavity present, according to the present invention. These results show that there is a very noticeable difference in the matching between antennae 1,1 and 2,2 as early as 1.4 mm in diameter, but with data analysis techniques, differences at much smaller cavity sizes are noticeable. Further tuning can make the sensitivity much higher to the exact center of the tooth.

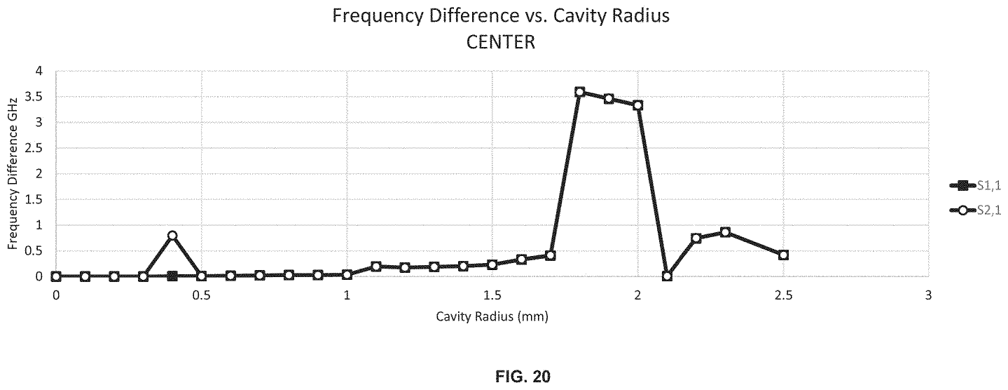

FIG. 20 is a graphical illustration of the results for the absolute value of the frequency response difference between FIG. 18 and the response of no cavity present, according to the present invention. This graph shows that the frequency sensitivity of the system matches very closely to that of the matching for a cavity in this location. Due to the center of the tooth being primarily utilized for the root of the tooth, sensitivity in this range can be useful to identifying issues with root decay and internal rotting.

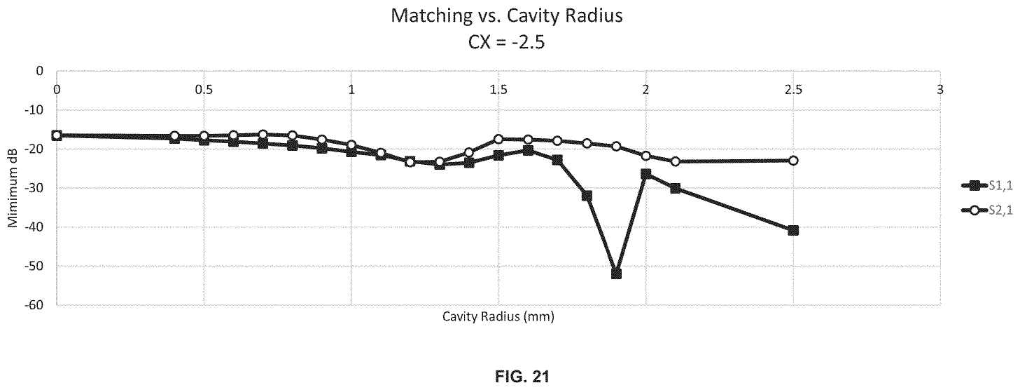

FIG. 21 is a graphical illustration of the results for the matching versus cavity radius when the sphere cavity's origin is shifted to −2.5 in the x direction (−2.5, 0, 0) with reference to FIG. 16, according to the present invention. The matching between the two antennae no longer matched, which signifies that the cavity is away from the center of the tooth. Significant difference between the two becomes evident at around 1 mm in diameter which is about 10% the total width of the simulated tooth.

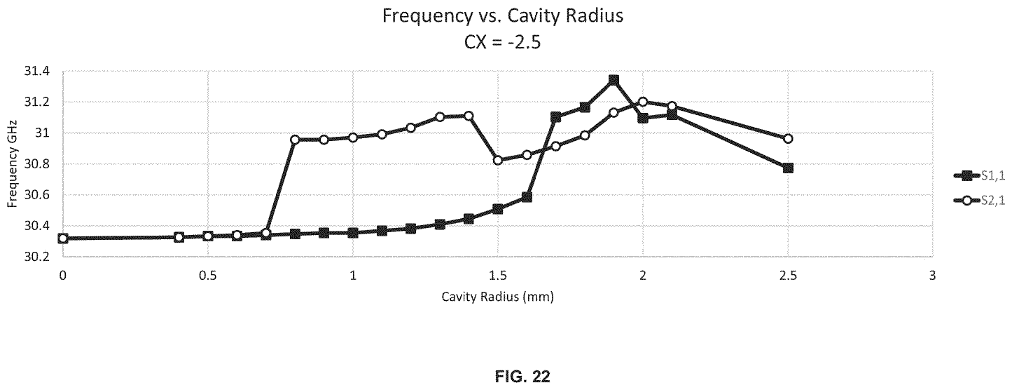

FIG. 22 is a graphical illustration of the results for frequency versus cavity radius when the sphere cavity's origin is shifted to −2.5 in the x direction (−2.5, 0, 0) with reference to FIG. 16, according to the present invention. The frequency response of antenna 2,2 is a lot more sensitive than that of antenna 1,1 which can help tell locationally where the cavity is in the tooth.

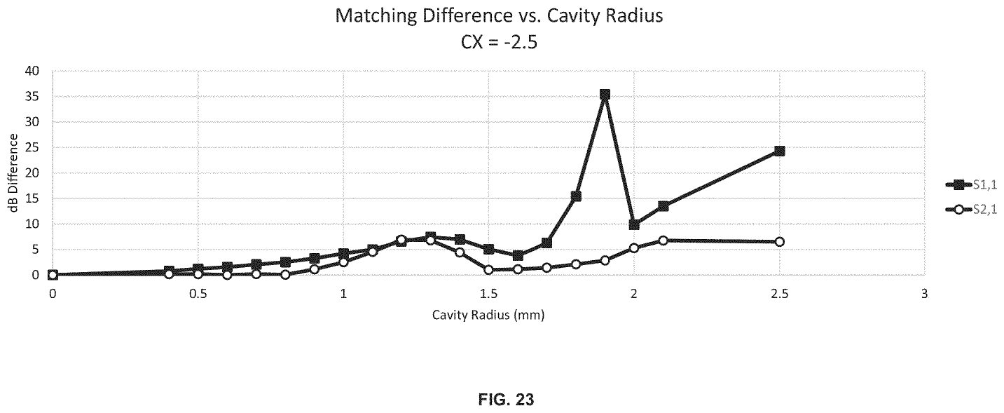

FIG. 23 a graphical illustration of the results for the absolute value of the matching difference between FIG. 21 and the response of no cavity present, according to the present invention. The absolute value of the matching shows that antenna 1 is more sensitive than antenna 2 when in the −X direction. Both antennae are able to see imperfections from there being no cavity.

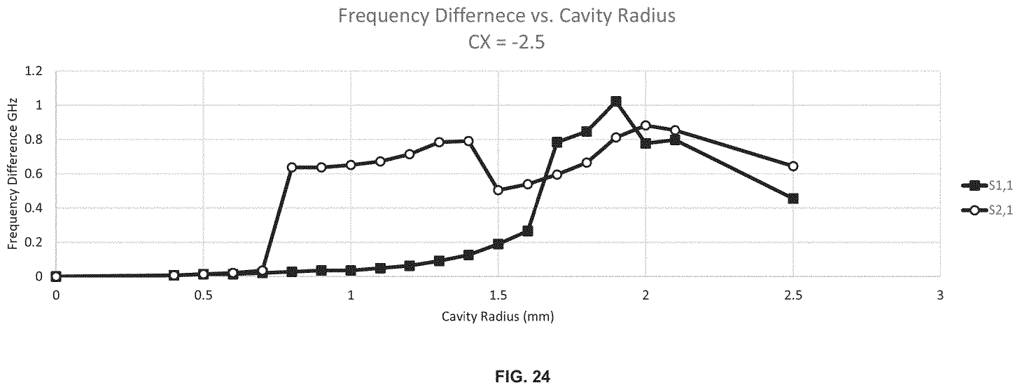

FIG. 24 a graphical illustration of the results for the absolute value of the frequency response difference between FIG. 22 and the response of no cavity present, according to the present invention. The frequency response shows that antenna 2 is more sensitive to the cavity in the −X direction than antenna 1, which is the opposite of what the matching results are.

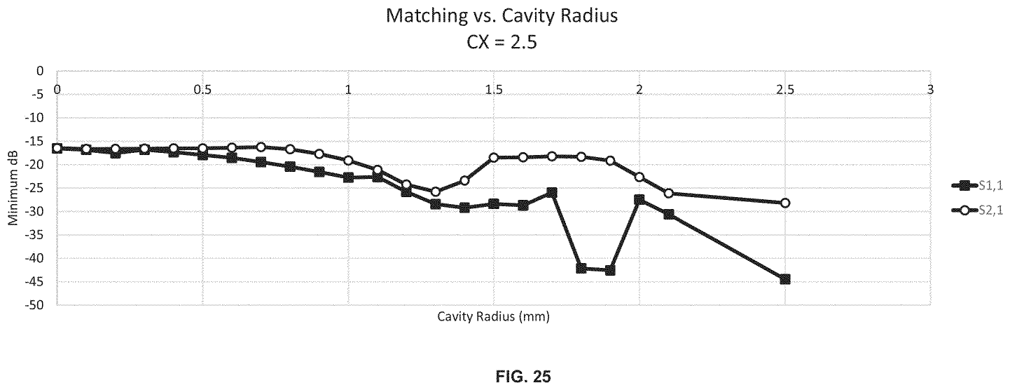

FIG. 25 is a graphical illustration of the results for the matching versus cavity radius when the sphere cavity's origin is shifted to +2.5 in the x direction (2.5, 0, 0) with reference to FIG. 16, according to the present invention. Notice how the response is almost the same as FIG. 21. This is important to be able to show position and size of the cavity; however, note that they are not the exact same value.

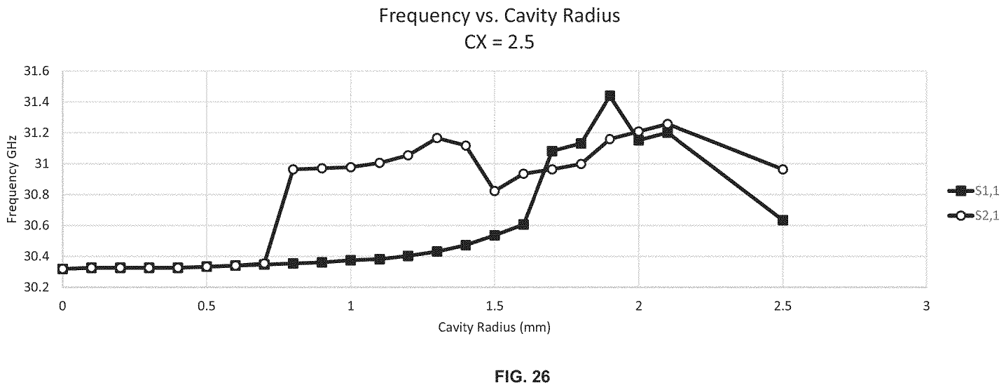

FIG. 26 is a graphical illustration of the results for the frequency versus cavity radius when the sphere cavity's origin is shifted to +2.5 in the x direction (2.5, 0, 0) with reference to FIG. 16, according to the present invention. Notice how the response is almost the same as FIG. 22. This is important to be able to show position and size of the cavity; however, note that they are not the exact same value.

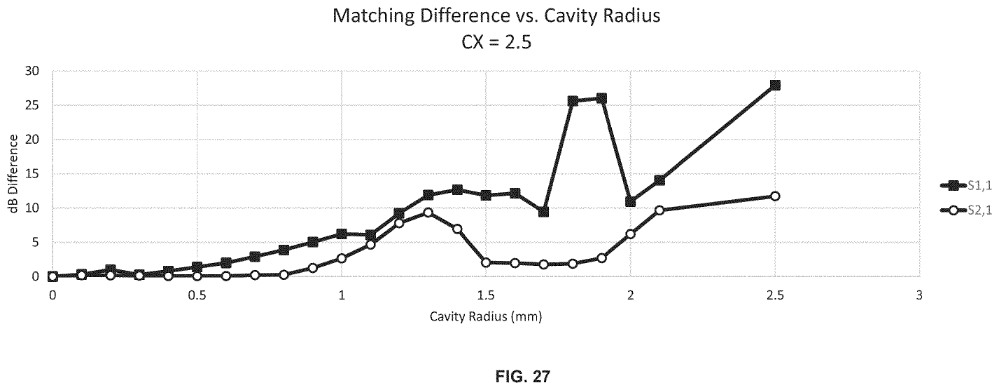

FIG. 27 is a graphical illustration of the results for the absolute value of the matching difference between FIG. 25 and the response of no cavity present, according to the present invention. This figure shows that the system becomes very sensitive to changes at an even smaller size of the cavity than in the −X direction.

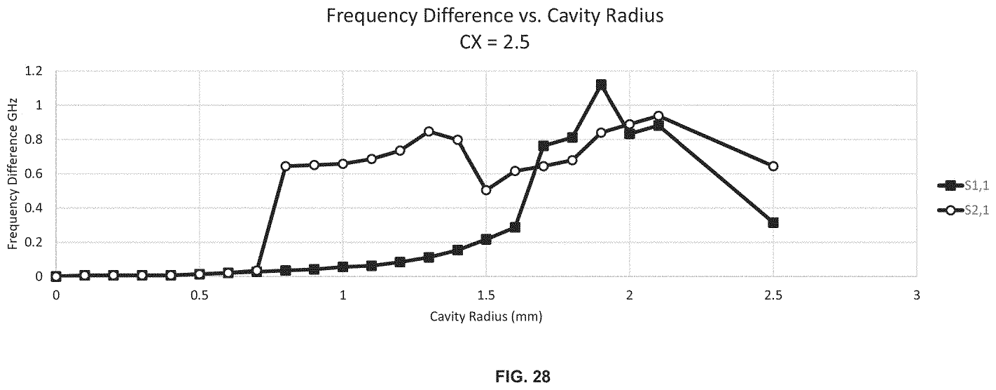

FIG. 28 is a graphical illustration of the results for the absolute value of the frequency response difference between FIG. 26 and the response of no cavity present, according to the present invention. The values in the smaller cavity range looks overlapped at this scale, but with the frequency being so high, the actual differences between the signals are still substantial. This graph shows substantial frequency differences between the two antennae at about 1.2 mm in diameter.

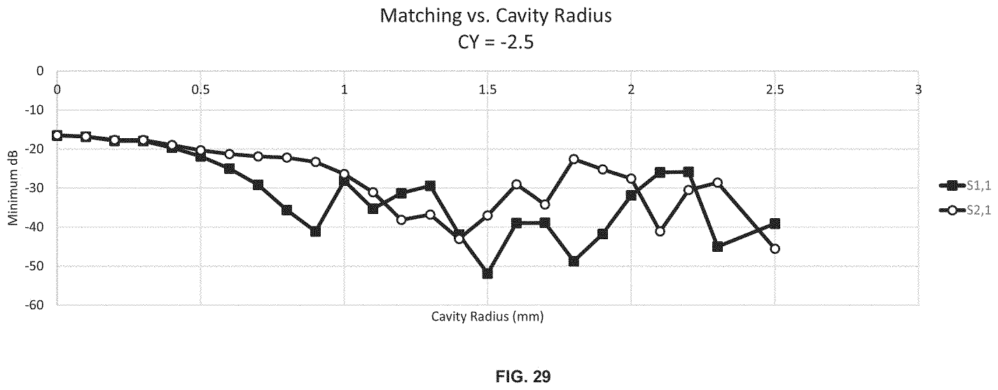

FIG. 29 is a graphical illustration of the results for the matching versus cavity radius when the sphere cavity's origin is shifted to −2.5 in the y direction (0, −2.5, 0) with reference to FIG. 16, according to the present invention. These results show a much greater difference between the antenna than when the cavity was shifted in the X direction. These results are useful when trying to determine which antenna the cavity is closer toward.

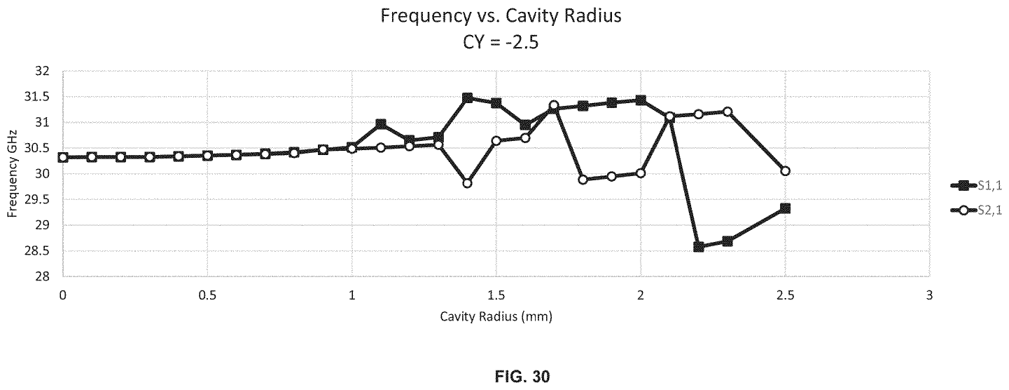

FIG. 30 is a graphical illustration of the results for the frequency versus cavity radius when the sphere cavity's origin is shifted to −2.5 in the y direction (0, −2.5, 0) with reference to FIG. 16, according to the present invention. The frequency response is very similar until the cavity becomes substantially large, but the differences from a smaller frequency scale are useful when determining more information about the cavity.

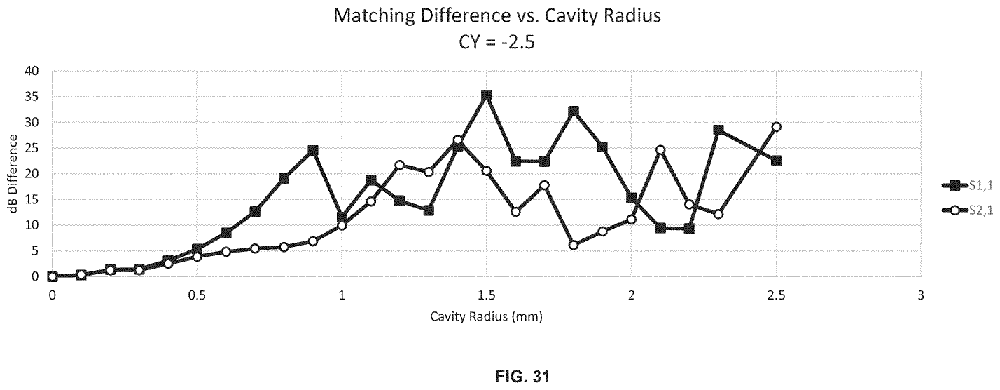

FIG. 31 is a graphical illustration of the results for the absolute value of the matching difference between FIG. 29 and the response of no cavity present, according to the present invention. These results are able to show the start of a cavity in the very early stages. Whenever the cavity is closer to an antenna, the matching to that antenna will become greater affected.

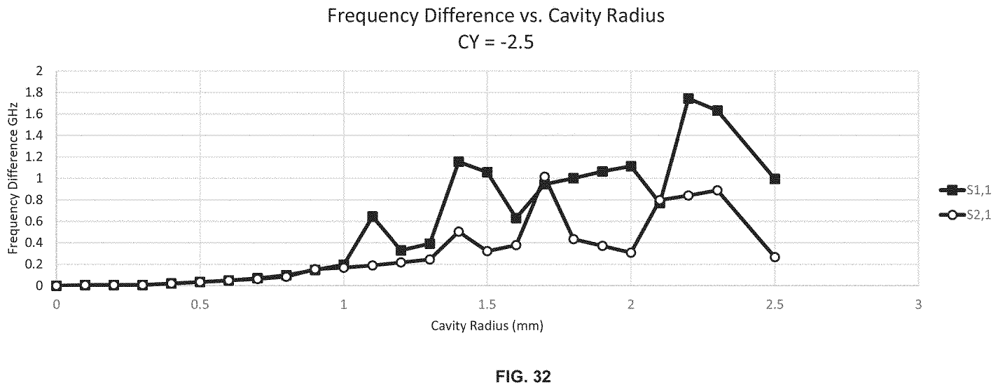

FIG. 32 is a graphical illustration of the results for the absolute value of the frequency response difference between FIG. 30 and the response of no cavity present, according to the present invention. These results once again show that the system does not appear to be very frequency sensitive to smaller cavities; however, closer inspection of the data shows that there are significant differences between the values even in very small cavity ranges. Another reason why working in a higher frequency range is more desirable for an application such as this.

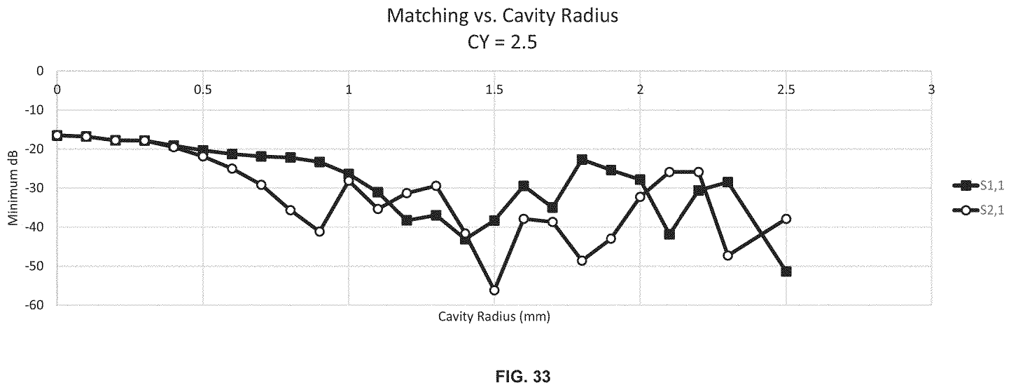

FIG. 33 is a graphical illustration of the results for the matching versus cavity radius when the sphere cavity's origin is shifted to +2.5 in the y direction (0, 2.5, 0) with reference to FIG. 16, according to the present invention. It should be noted how the response is almost the same as FIG. 29 except S1,1 and S2,2 are mirrored. This is important to be able to show position and size of the cavity; however, note that they are not the exact same value. The exact flip in the response is important to determining the side of the tooth the cavity is located in.

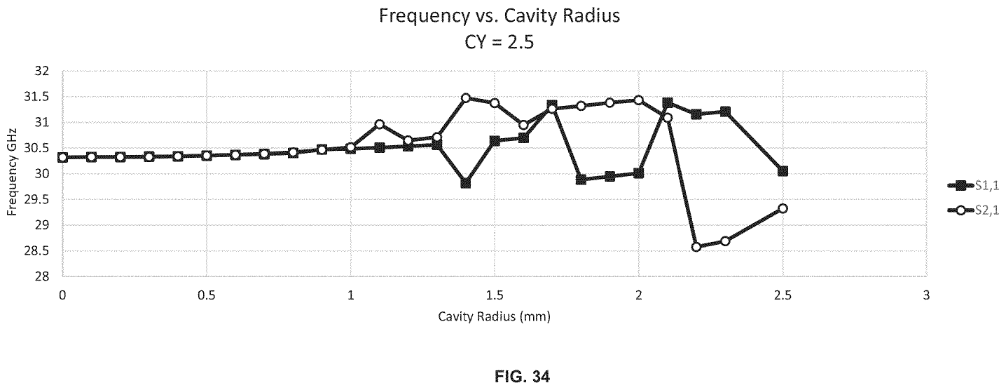

FIG. 34 is a graphical illustration of the results for the frequency versus cavity radius when the sphere cavity's origin is shifted to +2.5 in the y direction (0, 2.5, 0) with reference to FIG. 16, according to the present invention. It should be noted how the response is almost the same as FIG. 30 except S1,1 and S2,2 are mirrored. This is important to be able to show position and size of the cavity; however, note that they are not the exact same value.

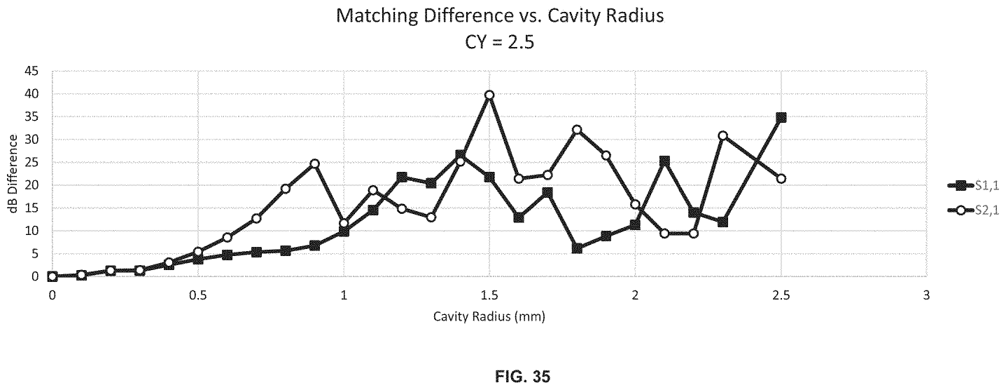

FIG. 35 is a graphical illustration of the results for the absolute value of the matching difference between FIG. 33 and the response of no cavity present, according to the present invention. This graph once again shows that the matching difference to there being no cavity signifies that the system is sensitive to abnormalities in extremely early stages of cavity formation.

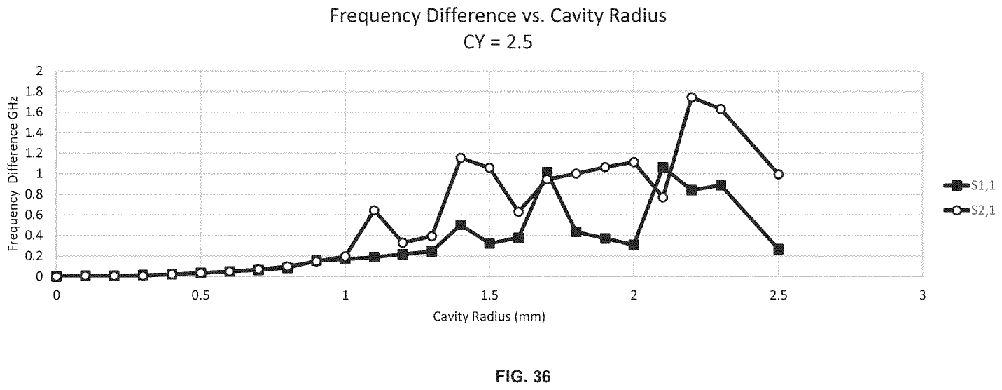

FIG. 36 is a graphical illustration of the results for the absolute value of the frequency response difference between FIG. 34 and the response of no cavity present, according to the present invention. The exact flipping of the results from − to + sides of the tooth are useful for showing the location of the cavity, and the overall size of the imperfection. The system can be tuned for even smaller analysis.

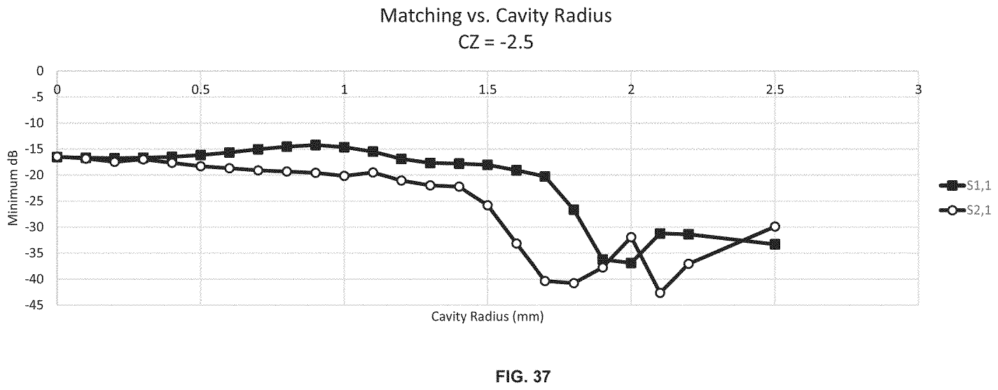

FIG. 37 is a graphical illustration of the results for the matching versus cavity radius when the sphere cavity's origin is shifted to −2.5 in the z direction (0, 0, −2.5) with reference to FIG. 16, according to the present invention. This graph signifies that the system is sensitive to cavities deep into the tooth, near the root, and completely invisible to external analysis methods. This makes the system extremely useful for early root issue detection.

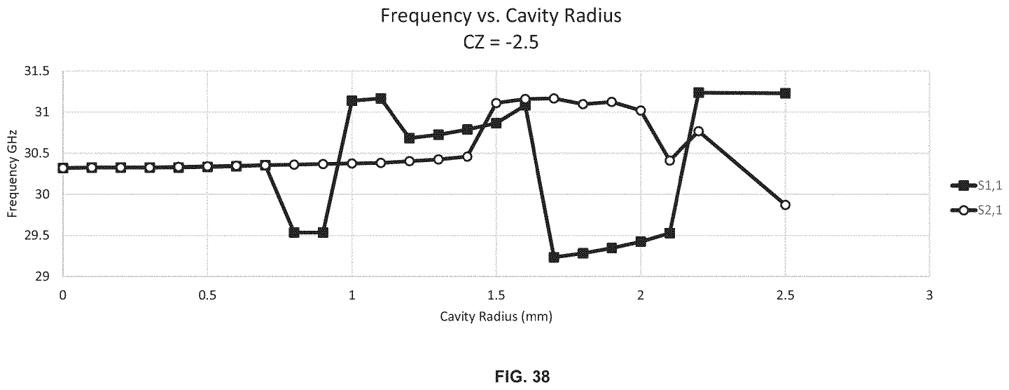

FIG. 38 is a graphical illustration of the results for the frequency versus cavity radius when the sphere cavity's origin is shifted to −2.5 in the z direction (0, 0, −2.5) with reference to FIG. 16, according to the present invention. This graph signifies that antenna 1 is far more sensitive to the depth of the cavity than antenna 2 is, which makes sense due to the polarity of the antenna in this simulation.

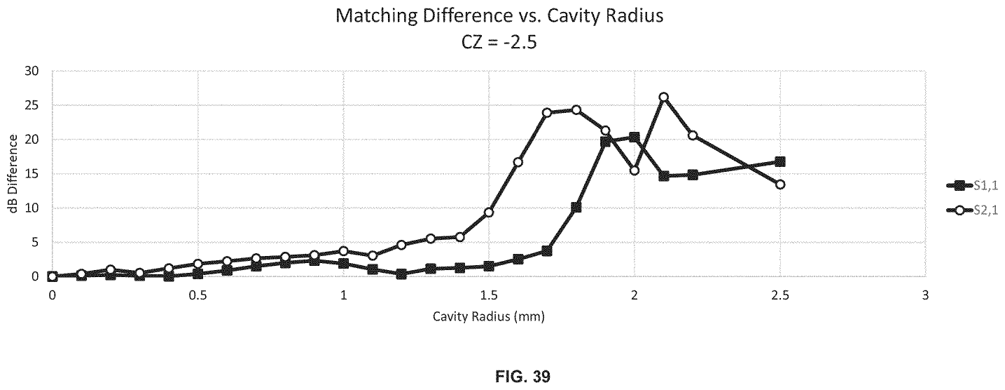

FIG. 39 is a graphical illustration of the results for the absolute value of the matching difference between FIG. 37 and the response of no cavity present, according to the present invention. These results show that when compared to a baseline of no cavity, the system is sensitive to cavities even deep into the tooth. A visual inspection would be blind to a cavity in this location. This becomes useful in early detection of root issues.

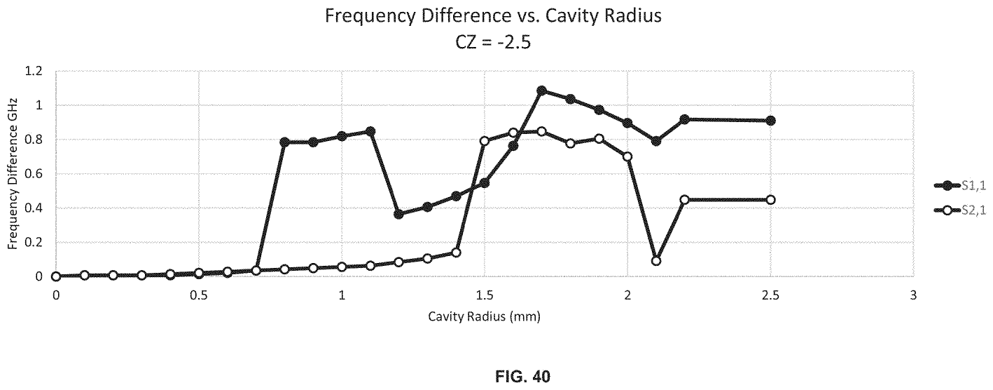

FIG. 40 is a graphical illustration of the results for the absolute value of the frequency response difference between FIG. 38 and the response of no cavity present, according to the present invention. This graph shows that as the cavity gets larger, antenna 1 becomes much more sensitive to it than antenna 2. The frequency range is still analyzable as the initial frequency is so high.

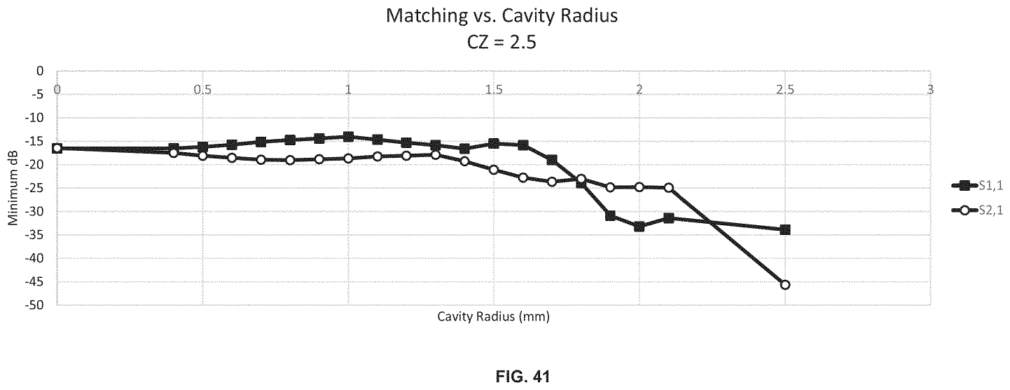

FIG. 41 is a graphical illustration of the results for the matching versus cavity radius when the sphere cavity's origin is shifted to +2.5 in the z direction (0, 0, 2.5) with reference to FIG. 16, according to the present invention. This result shows that the cavity towards the top of the tooth is also detectable by the system. It is to be noted how the response is similar to FIG. 37, but not as similar as the related figures in the x and y directions. This is important to be able to show position and size of the cavity.

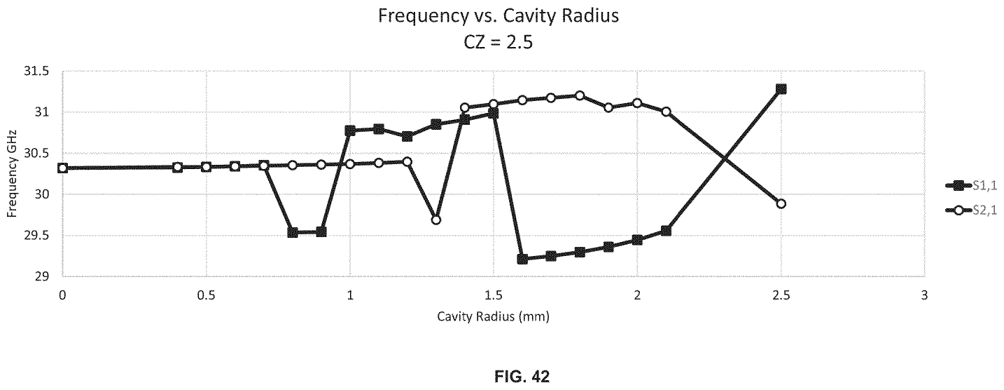

FIG. 42 is a graphical illustration of the results for the frequency versus cavity radius when the sphere cavity's origin is shifted to +2.5 in the z direction (0, 0, 2.5) with reference to FIG. 16, according to the present invention. The frequency response of the system is also shows to be rather sensitive to the relative size of the cavity. It is to be noted how the response is similar to FIG. 38, but not as similar as the related figures in the x and y directions. This is important to be able to show position and size of the cavity.

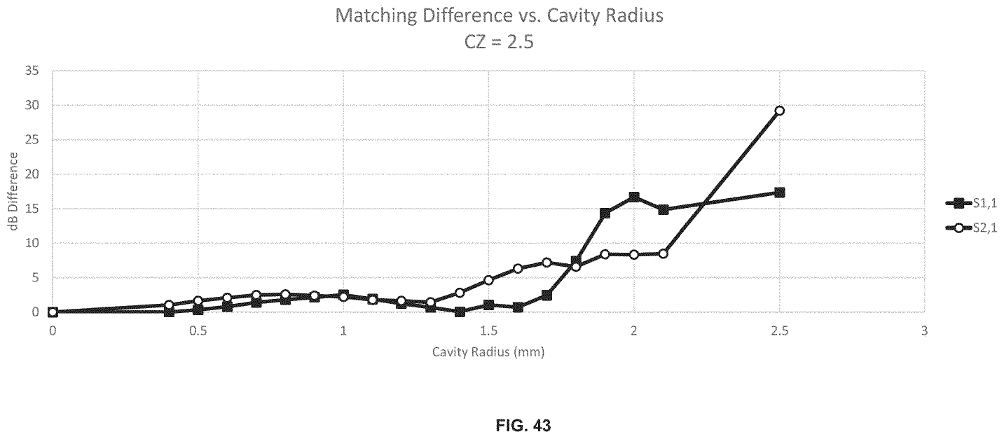

FIG. 43 is a graphical illustration of the results for the absolute value of the matching difference between FIG. 41 and the response of no cavity present, according to the present invention. This difference to a baseline of no cavity shows that the system is sensitive to changes as cavities work closer to the top of the tooth. It is to be noted the lack of similarities to FIG. 39. These are important results to show cavity location in the tooth.

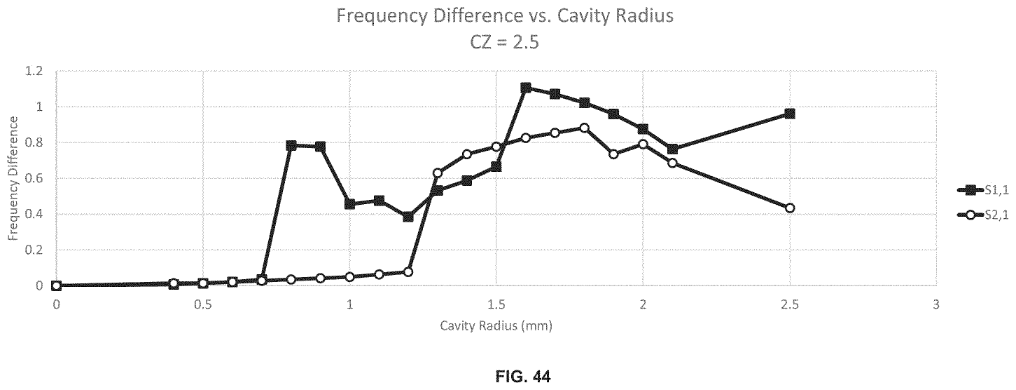

FIG. 44 is a graphical illustration of the results for the absolute value of the frequency response difference between FIG. 42 and the response of no cavity present, according to the present invention. The frequency response of the system is drastic once the cavity starts getting relatively large, but when seen on a smaller frequency scale, the results are more telling. It is to be noted the similarities of FIG. 44 to FIG. 40.

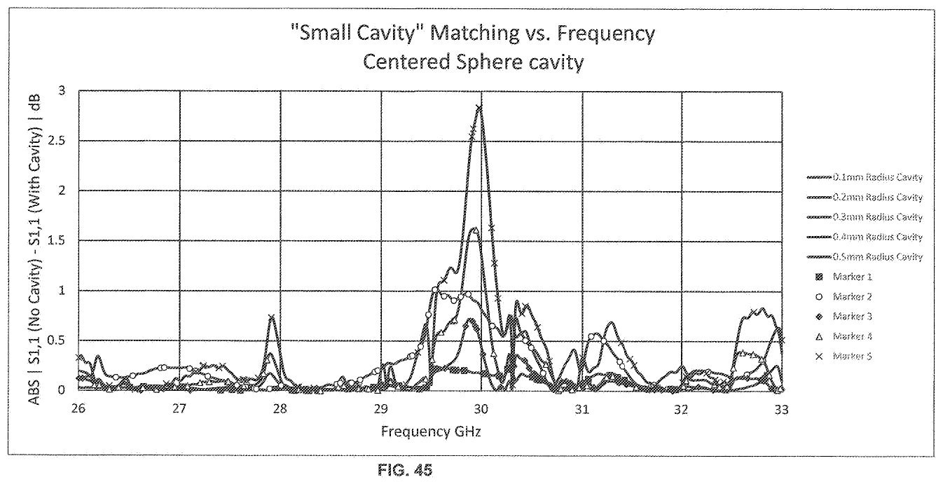

FIG. 45 is a graphical illustration of the absolute value of the matching difference of small cavities and no cavities versus frequency response when the cavity is centered in the middle of the tooth. The graph shows that there is a substantial difference between there being no cavity, and a cavity as small as 0.2 mm in diameter.

The preceding merely illustrates the principles of the invention. It will thus be appreciated that those skilled in the art will be able to devise various arrangements which, although not explicitly described or shown herein, embody the principles of the invention and are included within its spirit and scope. Furthermore, all examples and conditional language recited herein are principally intended expressly to be only for pedagogical purposes and to aid the reader in understanding the principles of the invention and the concepts contributed by the inventors to furthering the art and are to be construed as being without limitation to such specifically recited examples and conditions. Moreover, all statements herein reciting principles, aspects, and embodiments of the invention, as well as specific examples thereof, are intended to encompass both structural and functional equivalents thereof. Additionally, it is intended that such equivalents include both currently known equivalents and equivalents developed in the future, i.e., any elements developed that perform the same function, regardless of structure.

This description of the exemplary embodiments is intended to be read in connection with the figures of the accompanying drawing, which are to be considered part of the entire written description. In the description, relative terms such as “lower,” “upper,” “horizontal,” “vertical,” “above,” “below,” “up,” “down,” “top” and “bottom” as well as derivatives thereof (e.g., “horizontally,” “downwardly,” “upwardly,” etc.) should be construed to refer to the orientation as then described or as shown in the drawing under discussion. These relative terms are for convenience of description and do not require that the apparatus be constructed or operated in a particular orientation. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety.

The applicant reserves the right to physically incorporate into this specification any and all materials and information from any such patents, publications, scientific articles, web sites, electronically available information, and other referenced materials or documents to the extent such incorporated materials and information are not inconsistent with the description herein.

All of the features disclosed in this specification may be combined in any combination. Thus, unless expressly stated otherwise, each feature disclosed is only an example of a generic series of equivalent or similar features.

The specific methods and compositions described herein are representative of preferred embodiments and are exemplary and not intended as limitations on the scope of the invention. Other objects, aspects, and embodiments will occur to those skilled in the art upon consideration of this specification and are encompassed within the spirit of the invention. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, the terms “comprising”, “including”, “containing”, etc. are to be read expansively and without limitation. The methods and processes illustratively described herein suitably may be practiced in differing orders of steps, and that they are not necessarily restricted to the orders of steps indicated herein.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention. Thus, it will be understood that although the present invention has been specifically disclosed by various embodiments and/or preferred embodiments and optional features, any and all modifications and variations of the concepts herein disclosed that may be resorted to by those skilled in the art are considered to be within the scope of this invention.

The invention has been described broadly and generically herein. Each of the narrower species and sub-generic groupings falling within the generic disclosure also form part of the invention. This includes the generic description of the invention with a proviso or negative limitation removing any subject matter from the genus, regardless of whether or not the excised material is specifically recited herein.

Other modifications and implementations will occur to those skilled in the art without departing from the spirit and the scope of the invention. Accordingly, the description hereinabove is not intended to limit the invention.

Therefore, provided herein is a new and improved system and method for detecting dental anomalies, which according to various embodiments of the present invention, offers the following advantages: ease of use; the use of millimeter wave antennas; the elimination of exposure to X-rays; reduced cost; improved precision in anomalies detection; and the ability to detect dental anomalies.

In fact, in many of the preferred embodiments, these advantages of ease of use, the use of millimeter wave antennas, the elimination of exposure to X-rays, reduced cost, improved precision in anomalies detection, and the ability to detect dental anomalies are optimized to an extent that is considerably higher than heretofore achieved in prior, known systems and methods for detecting dental anomalies.

We claim:

1. A system for the detection of dental anomalies, comprising:
a casing, wherein the casing comprises;
a plurality of antenna sources located along one side of the casing, and
a plurality of sensors located along a second side of the casing;
a mouthpiece assembly substantially located between the first and second sides of the casing;
a power module operatively connected to each of the plurality of antenna sources for providing electrical power to each of the antenna sources; and
an analytics module operatively connected to each of the plurality of sensors for receiving and analyzing dental anomalies.

2. The system for the detection of dental anomalies, according to claim 1, wherein the casing is shaped to size and fit over a row of teeth.

3. The system for the detection of dental anomalies, according to claim 1, wherein the casing is constructed of a UV-resistant, durable, medical grade polymeric material.

4. The system for the detection of dental anomalies, according to claim 1, wherein each of the plurality of dipole antennas sources is further comprised of:
a radio frequency (RF) antenna.

5. The system for the detection of dental anomalies, according to claim 1, wherein each of the plurality of antennas sources is arranged located along one side of the casing in a triangular pattern.

6. The system for the detection of dental anomalies, according to claim 1, wherein each of the plurality of sensors is further comprised of:
a broadband millimeter wave (mmW) antenna that is capable of detecting frequencies between 3 GHz and 300 GHz.

7. The system for the detection of dental anomalies, according to claim 1, wherein the mouthpiece assembly further comprises:
a mouthpiece;
an electronics housing and reflective casing located adjacent to the mouthpiecee;
a top teeth moldable mouthpiece operatively connected to the electronics housing and reflective casing;
a bottom teeth moldable mouthpiece operatively connected to the electronics housing and reflective casing; and
a rear reflective casing operativley connected to the top teeth moldable mouthpiece and the bottom teeth moldable mouthpiece.

8. A method of constructing a system for the detection of dental anomalies, comprising:
providing a casing, wherein the casing comprises;
a plurality of antenna sources located along one side of the casing, and
a plurality of sensors located along a second side of the casing;
providing a mouthpiece assembly substantially located between the first and second sides of the casing;
providing a power module operatively connected to each of the plurality of antenna sources for providing electrical power to each of the antenna sources; and
providing an analytics module operatively connected to each of the plurality of sensors for receiving and analyzing dental anomalies.

9. The method, according to claim 8, wherein the casing is shaped to size and fit over a row of teeth.

10. The method, according to claim 8, wherein the casing is constructed of a UV-resistant, durable, medical grade polymeric material.

11. The method, according to claim 8, wherein each of the plurality of antennas sources is further comprised of:
a radio frequency (RF) antenna.

12. The method, according to claim 8, wherein each of the plurality of antennas sources is arranged located along one side of the casing in a triangular pattern.

13. The method, according to claim 8, wherein each of the plurality of sensors is further comprised of:
a broadband millimeter wave (mmW) antenna that is capable of detecting frequencies between 3 GHz and 300 GHz.

14. The method, according to claim 8, wherein the mouthpiece assembly is further comprised of:
providing a mouthpiece;
locating an electronics housing and reflective casing adjacent to the mouthpiece;
attaching a top teeth moldable mouthpiece to the electronics housing and reflective casing;
attaching a bottom teeth moldable mouthpiece to the electronics housing and reflective casing; and
attaching a rear reflective casing to the top teeth moldable mouthpiece and the bottom teeth moldable mouthpiece.

15. A method of using a system for the detection of dental anomalies, comprising:
providing a casing, wherein the casing comprises;
a plurality of antenna sources located along one side of the casing, and
a plurality of sensors located along a second side of the casing;
providing a mouthpiece assembly substantially located between the first and second sides of the casing;
providing a power module operatively connected to each of the plurality of antenna sources for providing electrical power to each of the antenna sources;
providing an analytics module operatively connected to each of the plurality of sensors for receiving and analyzing dental anomalies;
locating the casing and the mouthpiece in a user's mouth;
operating the power module operatively connected to each of the plurality of antenna sources to provide electrical power to each of the antenna sources; and
operating the analytics module operatively connected to each of the plurality of sensors to receive and analyze dental anomalies in the user's mouth.

16. The method, according to claim 15, wherein the casing is shaped to size and fit over a row of teeth.

17. The method, according to claim 15, wherein the casing is constructed of a UV-resistant, durable, medical grade polymeric material.

18. The method, according to claim 15, wherein each of the plurality of antenna sources is further comprised of:
a radio frequency (RF) antenna.

19. The method, according to claim 15, wherein each of the plurality of sensors is further comprised of:
a broadband millimeter wave (mmW) antenna that is capable of detecting frequencies between 3 GHz and 300 GHz·fff

20. The method, according to claim 15, wherein the mouthpiece assembly is further comprised of:
a mouthpiece;
an electronics housing and reflective casing located adjacent to the mouthpiece;
a top teeth moldable mouthpiece operatively connected to the electronics housing and reflective casing;
a bottom teeth moldable mouthpiece operatively connected to the electronics housing and reflective casing; and
a rear reflective casing operativley connected to the top teeth moldable mouthpiece and the bottom teeth moldable mouthpiece.
